(19) United States
(12) Patent Application Publication
(10) Pub. No.: US 2022/0283071 A1
(43) Pub. Date: Sep. 8, 2022

(54) SPERM SORTING APPARATUS AND SPERM SORTING METHOD

(71) Applicant: National Tsing Hua University, Hsinchu City (TW)

(72) Inventors: Fan-Gang Tseng, Hsinchu City (TW); Suei-Shen Wang, Hsinchu City (TW); I-Jui Chen, Hsinchu City (TW)

(73) Assignee: National Tsing Hua University, Hsinchu City (TW)

(21) Appl. No.: 17/373,785

(22) Filed: Jul. 13, 2021

(30) Foreign Application Priority Data
Mar. 2, 2021 (TW) 110107226

(51) Int. Cl. G01N 15/10; C12N 5/071; G01N 1/40

(52) U.S. Cl. CPC G01N 15/10; C12N 5/0612; G01N 1/4005; G01N 2015/0003

(57) ABSTRACT

A sperm sorting apparatus and a sperm sorting method are provided. The sperm sorting apparatus includes a medium chamber, a waste chamber and a sorting channel communicated with and extending along a first direction in between. The medium chamber is configured to contain a medium solution. The waste chamber disposed aside the medium chamber is configured to contain a residual solution after sorting. The residual solution includes low motility sperms and/or dead sperms. The sorting channel is configured to be inserted with a sperm sample, such that sperms in the sperm sample are sorted in corresponding to a medium solution flow from the medium chamber. The sorting channel has first and second portions. The first portion is closer to the medium chamber than the second portion. A width of the first portion measured along a second direction is greater than a critical dimension, ranging from 200 μm to 400 μm.

### CROSS-REFERENCE TO RELATED APPLICATION

This application claims the priority benefit of Taiwan application serial no. 110107226, filed on Mar. 2, 2021. The entirety of the above-mentioned patent application is hereby incorporated by reference herein and made a part of this specification.

### BACKGROUND

### Technical Field

The present invention relates to a sperm sorting apparatus and a sperm sorting method.

### Description of Related Art

Infertility has become one of the common problems in modern society. Various artificial fertilization methods have been developed for addressing such problem. For instance, current artificial fertilization methods include intrauterine insemination (IUI), in vitro fertilization (IVF) and intracytoplasmic sperm injection (ICSI) and so forth. These methods require high motility sperms for each test. Hence, a method for sorting out high motility sperms is important in the field of artificial fertilization. Currently, finding a method for sorting out great amount of the high motility sperms from a sperm sample is quite challenging.

### SUMMARY

In an aspect of the present invention, a sperm sorting apparatus is provided. The sperm sorting apparatus comprises: a medium chamber, configured to contain a medium solution; a waste chamber, disposed aside the medium chamber, and configured to contain a residual solution obtained after sorting, wherein the residual solution includes low motility sperms and/or dead sperms; and a sorting channel, extending between and communicated with the medium chamber and the waste chamber along a first direction, and configured to be inserted with a sperm sample, such that sperms in the sperm sample are sorted in corresponding to a medium solution flow entering the sorting channel from the medium chamber, wherein the sorting channel has a first portion and a second portion, the first portion is closer to the medium chamber than the second portion, a width of the first portion measured along a second direction is greater than a critical dimension, the second direction is intersected with the first direction, and the critical dimension ranges from 200 μm to 400 μm.

In some embodiments, sperm sorting apparatus further comprises parallel micro-channels, extending along the second direction and disposed between the medium chamber and the sorting channel.

In some embodiments, the first portions of the sorting channel converges toward the medium chamber, a width of at least a section of the first portion measured along the second direction is greater than the critical dimension.

In some embodiments, the sorting channel is communicated to outside of the sperm sorting apparatus through an input/output hole.

In some embodiments, the input/output hole is communicated with the first portion of the sorting channel.

In some embodiments, the second portion of the sorting channel converges toward the waste chamber.

In some embodiments, an end portion of the sorting channel communicated with the waste chamber is a narrow channel, and a width of the sorting channel measured along the second direction is at its minimum at the narrow channel.

In another aspect of the present invention, a sperm sorting method is provided. The sperm sorting method comprises: providing a sperm sorting apparatus, comprising a medium chamber, a waste chamber and a sorting channel extending between and communicated with the medium chamber and the waste chamber along a first direction, a width of at least a section of the sorting channel measured along a second direction is greater than a critical dimension, and the critical dimension ranges from 200 μm to 400 μm; inserting a medium solution to the medium chamber and the waste chamber, till liquid levels in the medium chamber, the sorting channel and the waste chamber are balanced; inserting a sperm sample into the sorting channel; additionally inserting a medium solution into the medium chamber, such that the medium solution in the medium chamber flows into the sorting channel to form a medium solution flow, wherein sperms in the sperm sample are sorted in corresponding to the medium solution flow; and extracting a sorted solution from the sorting channel.

In some embodiments, the sperm sorting method further comprises: rinsing the medium chamber, the sorting channel and the waste chamber with an additional medium solution before the step of inserting the medium solution to the medium chamber and the waste chamber.

In some embodiments, the sperm sorting method further comprises: intermittently repeating the step of additionally inserting a medium solution into the medium chamber before the step of extracting the sorted solution from the sorting channel.

The sperm sorting apparatus according to embodiments of the present invention utilizes both of a behavioral tendency that the high motility sperms swim against the medium solution flow and a behavioral tendency that the high motility sperms gather across the inner surface of the sorting channel, thus a great amount of high motility sperms can be extracted by using the sperm sorting apparatus. As a result, in addition to be applicable for the ICSI artificial fertilization method, the sperm sorting apparatus 10 may be further applicable for the WF artificial fertilization method. During a sperm sorting process, the high motility sperms at least mostly stay in the sorting channel since being inserted into the sorting channel, while the low motility and/or dead sperms are carried to the waste chamber along the medium solution flow. Subsequently, the sorted solution in the sorting channel may be extracted from the sorting channel. In other words, a moving distance of the high motility sperms is short, thus the sorting can be performed in a short time, and energy consumption of the high motility sperms can be low. Moreover, since the sperm sample is directly inserted into the sorting channel, substantially all of the sperms in the sperm sample can be ensured to be sorted, thus a great sorting efficiency can be promised.

To make the aforementioned more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1A is a three-dimensional perspective view schematically illustrating a sperm sorting apparatus according to some embodiments of the present invention.

FIG. 1B is a three-dimensional view schematically illustrating an assembled sperm sorting apparatus according to some embodiments of the present invention.

FIG. 2A is a plan view schematically illustrating a bottom surface of the top substrate as shown in FIG. 1A.

FIG. 2B is an enlarged three-dimensional view schematically illustrating the micro-channels as shown in FIG. 1A and FIG. 2A.

FIG. 3 is a cross-sectional view schematically illustrating a sorting channel in a sperm sorting apparatus according to some embodiments of the present invention.

FIG. 4 is a flow diagram illustrating a sperm sorting method according to some embodiments of the present invention.

### DESCRIPTION OF THE EMBODIMENTS

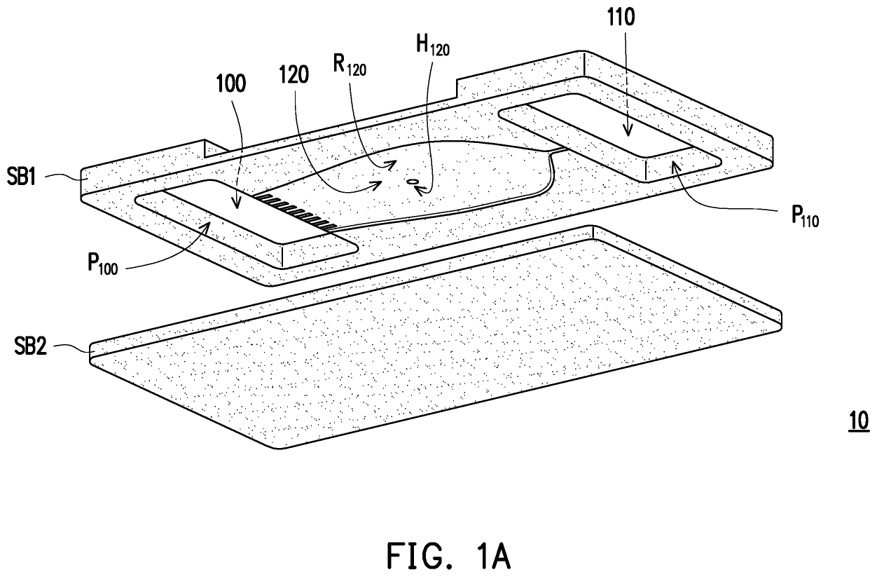

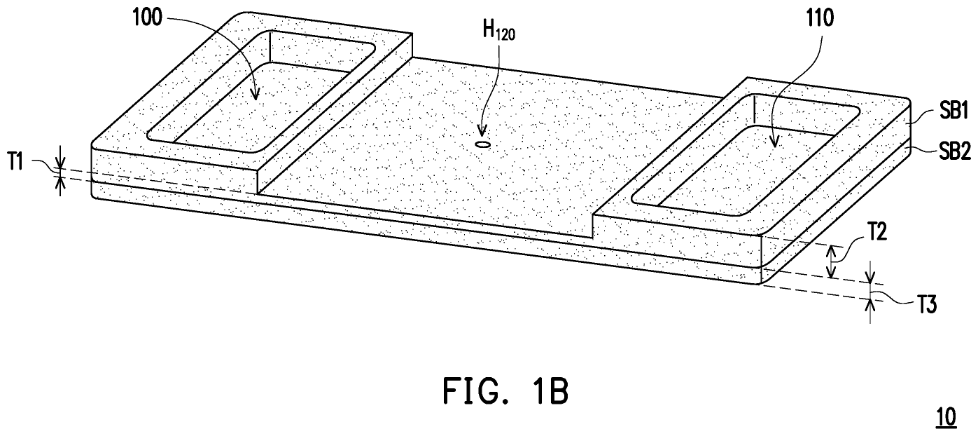

FIG. 1A is a three-dimensional perspective view schematically illustrating a sperm sorting apparatus 10 according to some embodiments of the present invention. FIG. 1B is a three-dimensional view schematically illustrating the sperm sorting apparatus 10 in an assembled state, according to some embodiments of the present invention.

Referring to FIG. 1A, the sperm sorting apparatus 10 includes a medium chamber 100, a waste chamber 110 and a sorting channel 120 extending between and communicated to the medium chamber 100 and the waste chamber 110. The medium chamber 100 is configured to contain a medium solution. As will be further described, high motility sperms in a sperm sample inserted into the sorting channel 120 swim against the medium solution flowing into the sorting channel 120 from the medium chamber 100, and stay in the sorting channel 120, to be extracted. On the other hand, a residual solution obtained after sorting and including low motility and/or dead sperms flow into the waste chamber 110 along the sorting channel 120, and can be collected. In this way, the sperm sample can be sorted by the sperm sorting apparatus 10.

In some embodiments, the sperm sorting apparatus 10 is formed by attaching a top substrate SB1 with a bottom substrate SB2. In these embodiments, the bottom substrate SB2 may be a flat plate, and the top substrate SB1 may have openings and a recess at its bottom surface. For instance, the openings of the top substrate SB1 may include an opening P₁₀₀ for forming the medium chamber 100 after the top substrate SB1 is attached with the bottom substrate SB2, and may include an opening P₁₁₀ for forming the waste chamber 110 after the top substrate SB1 is attached with the bottom substrate SB2. In addition, the recess of the top substrate SB1 may include a recess R₁₂₀ for forming the sorting channel 120 after the top substrate SB1 is attached with the bottom substrate SB2. A portion of the bottom substrate SB2 overlapped with the opening P₁₀₀ may define a bottom surface of the medium chamber 100, and a sidewall of the opening P₁₀₀ may define a sidewall of the medium chamber 100. A portion of the bottom substrate SB2 overlapped with the opening P₁₁₀ may define a bottom surface of the waste chamber 110, and a sidewall of the opening P₁₁₀ may define a sidewall of the waste chamber 110. In addition, a portion of the bottom substrate SB2 overlapped with the recess R₁₂₀ may define a bottom surface of the sorting channel 120, and a surface of the recess R₁₂₀ (i.e., the recessed surface at the bottom surface of the top substrate SB1) may define a top surface and a sidewall of the sorting channel 120.

The sperm sorting apparatus 10 may further include an input/output hole H₁₂₀ communicated with the sorting channel 120. The sperm sample may be inserted into the sorting channel 120 from outside of the sperm sorting apparatus 10 through the input/output hole H₁₂₀, and the high motility sperms from the sperm sample may be extracted from the sorting channel 120 through the input/output hole H₁₁₀. In those embodiments where the sperm sorting apparatus 10 is formed by attaching the top substrate SB1 with the bottom substrate SB2, the input/output hole H₁₁₀ penetrates through a portion of the top substrate SB1 having the recess R₁₂₀, so as to communicate with the sorting channel 120 defined by the recess R₁₂₀ of the top substrate SB1 and the portion of the bottom substrate SB2 overlapped with the recess R₁₂₀.

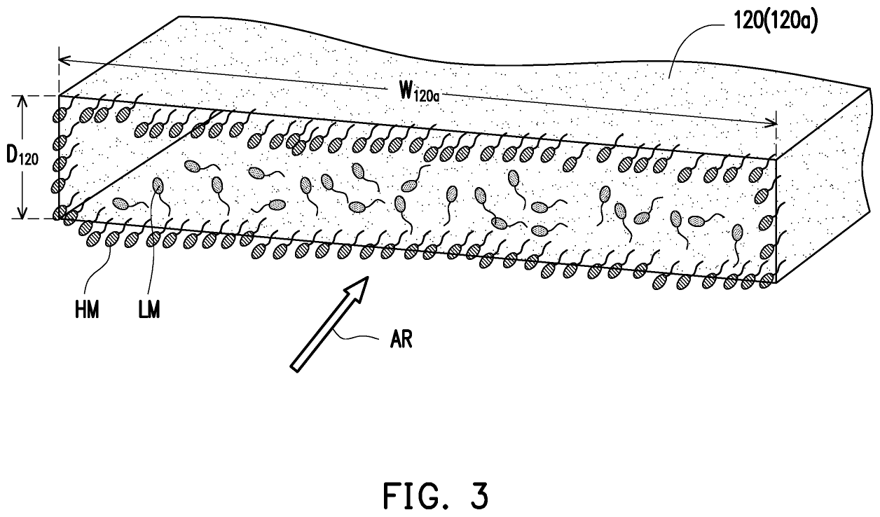

Referring to FIG. 1B, after the top substrate SB1 is attached with the bottom substrate SB2, the medium chamber 100 and the waste chamber 110 are defined. However, since the sorting channel 120 may be an inner space in the sperm sorting apparatus 10, the sorting channel 120 may not be observed from the appearance of the sperm sorting apparatus 10, except for the input/output hole H₁₂₀ communicated to the sorting channel 120. In some embodiments, a thickness T1 of the portion of the top substrate SB1 having the recess R₁₂₀ is less than a thickness T2 of the portions of the top substrate SB1 having the openings P₁₀₀, P₁₁₀. The thickness T1 may be slightly greater than a height D₁₂₀ of the sorting channel 120 as will be described with reference to FIG. 3. Further, in these embodiments, a ratio of the thickness T2 with respect to the thickness T1 may range from, for example, 4 to 5. For instance, the thickness T2 may range from 4 mm to 20 mm, while the thickness T1 may range from 1 mm to 4 mm. On the other hand, in some embodiments, the bottom substrate SB2 has a consistent thickness T3. For instance, the thickness T3 may range from 1 mm to 4 mm.

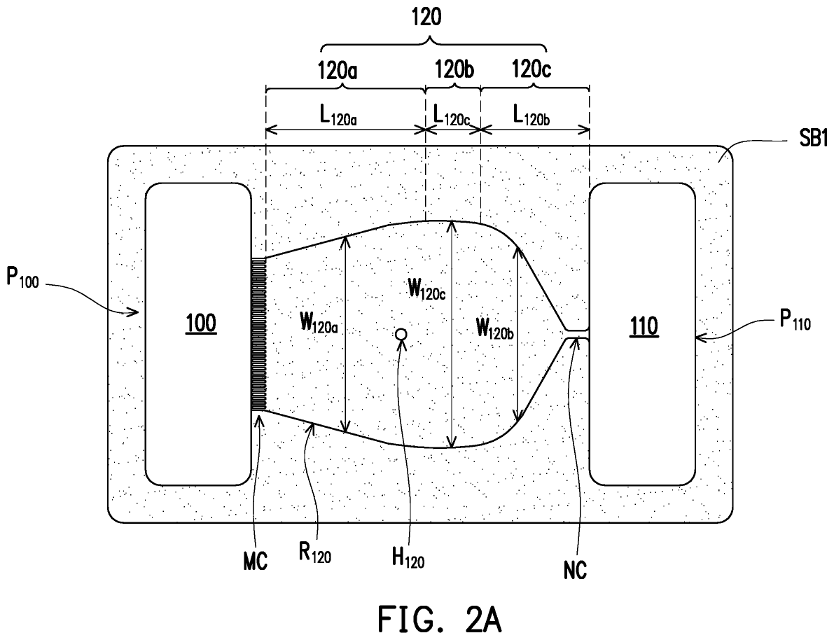

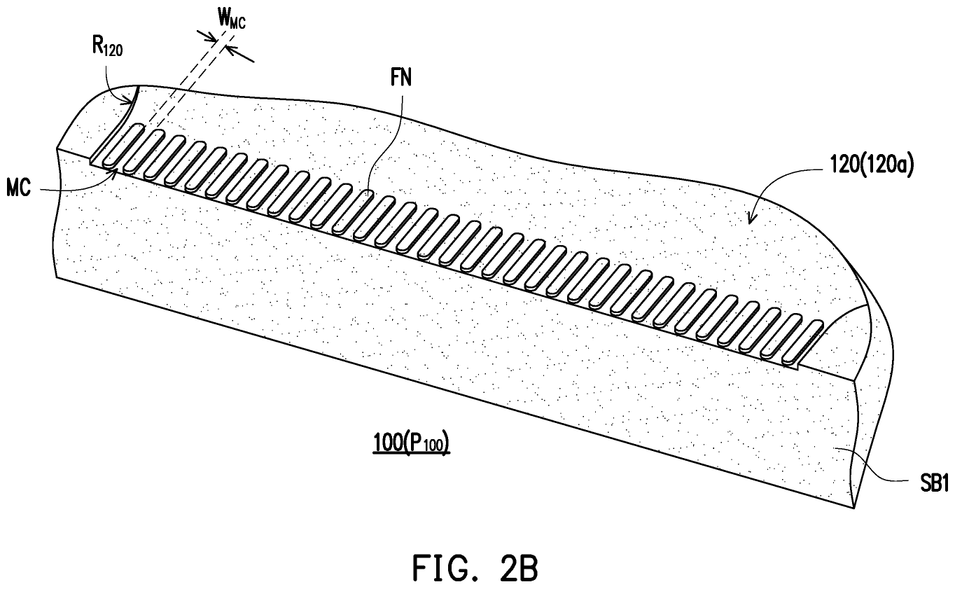

FIG. 2A is a plan view schematically illustrating the bottom surface of the top substrate SB1 as shown in FIG. 1A. FIG. 2B is an enlarged three-dimensional view schematically illustrating micro-channels MC as shown in FIG. 1A and FIG. 2A.

Referring to FIG. 1A and FIG. 2A, the sorting channel 120 may have a first portion 120a close to the medium chamber 100, and may have a second portion 120b close to the waste chamber 110. The first portion 120a of the sorting channel 120 may converge toward the medium chamber 100, while the second portion 120b of the sorting channel 120 may converge toward the waste chamber 110. In other words, a width W₁₂₀ₐ of the first portion 120a may gradually decrease toward the medium chamber 100, and a width W_{120b} of the second portion 120b may gradually decrease toward the waste chamber 110. The high motility sperms have a behavioral tendency of swimming against flow (e.g., a medium solution flow), such that the high motility sperms may gather in a region where a flow velocity is relatively high. By having the portion of the sorting channel 120 close to the medium chamber 100 (i.e., the first portion 120a) converges toward the medium chamber 100, the medium solution may enter the sorting channel 120 with a relatively high flow velocity, and may gradually slow down while away from the medium chamber 100. In this way, the high motility sperms may gather in the portion of the sorting channel 120 close to the medium chamber 100 (i.e., the first portion 120a), whereas low motility and/or dead sperms may move toward the waste chamber 110 along the medium solution flow. In addition, by having the portion of the sorting channel 120 close to the waste chamber 110 (i.e., the second portion 120b) converges toward the waste chamber 110, the medium solution flow that carries the low motility and/or dead sperms and leaves for the waste chamber 110 from the sorting channel 120 may be accelerated. In some embodiments, an end portion of the second portion 120b of the sorting channel 120 (i.e., an end of the sorting channel 120 directly communicated with the waste chamber 110) is designed as a narrow channel NC, such that the medium solution flow that carries the low motility and/or dead sperms may enter the waste chamber 110 with a further accelerated flow velocity. In these embodiments, the narrow channel NC may be a necking portion of the sorting channel 120, and the width W_{120b} is significantly reduced to a minimum at the narrow channel NC. Moreover, in some embodiments, a convergence rate of the first portion 120a of the sorting channel 120 is less than a convergence rate of the second portion 120b of the sorting channel 120. In other words, a reduction rate of the width W₁₂₀ₐ may be less than a reduction rate of the width W_{120b}. By such design, the medium solution flow from the medium chamber 100 may be avoided from entering the sorting channel 120 with an excessively high flow velocity, which may result in carrying the high motility sperms to the portion of the sorting channel 120 close to the waste chamber 110 (i.e., the second portion 120b). For instance, the width W₁₂₀ₐ may range from 20 mm to 30 mm. In addition, the width W_{120b} may range from 1 mm to 28 mm, and the width W_{120b} may be 1 mm at the narrow channel NC. On the other hand, a length L₁₂₀ₐ of the first portion 120a may range from 17 mm to 20 mm, while a length L_{120b} of the second portion 120b may range from 15 mm to 18 mm.

In some embodiments, the input/output hole H₁₂₀ is communicated with the first portion 120a of the sorting channel 120, where the high motility sperms swim against the medium solution flow and gather. Accordingly, more of the high motility sperms can be extracted from the input/output hole H₁₂₀. However, the input/output hole H₁₂₀ should be laterally spaced apart from the medium chamber 100 by an appropriate distance, in order to prevent the sperms in the sperm sample inserted into the sorting channel 120 through the input/output hole H₁₂₀ from flowing back to the medium chamber 100. For instance, the input/output hole H₁₂₀ may be close to a side of the first portion 120a that is away from the medium chamber 100.

In some embodiments, the sorting channel 120 further has a third portion 120c between the first portion 120a and the second portion 120b. A width W_{120c} of the third portion 120c may be substantially equal to a maximum value of the width W₁₂₀ₐ of the first portion 120a and a maximum value of the width W_{120b} of the second portion 120b. For instance, the width W_{120c} of the third portion 120c of the sorting channel 120 may range from 28 mm to 30 mm. Further, a length L_{120c} of the third portion 120c of the sorting channel 120 may range from 9 mm to 12 mm.

Referring to FIG. 2A and FIG. 2B, in some embodiments, the first portion 120a of the sorting channel 120 is communicated with the medium chamber 100 through parallel micro-channels MC. A width W_{MC} of each micro-channel MC (shown in FIG. 2B) is significantly less than a width of the sorting channel 120 (e.g., the width W₁₂₀ₐ as described with reference to FIG. 2A), in order to prevent the high motility sperms gathering in the first portion 120a of the sorting channel 120 from flowing back to the medium chamber 100 through the micro-channels MC. For instance, the width W_{MC} of each micro-channel MC may range from 0.2 mm to 0.4 mm. In some embodiments, the micro-channels MC are defined by fin structures FN at the bottom surface of the top substrate SB1. The fin structures FN protrude from the recessed surface of the recess R₁₂₀, and are arranged side-by-side between the medium chamber 100 and a side of the sorting channel 120. Each of the fin structures FN may be located between adjacent micro-channels MC. In addition, after the top substrate SB1 is attached with the bottom substrate SB2 (as shown in FIG. 1B), the fin structures FN may contact the bottom substrate SB2, such that the medium chamber 100 may be communicated with the sorting channel 120 only through the micro-channels MC. Accordingly, a height of the fin structures FN may be substantially equal to a depth of the recess R₁₂₀.

FIG. 3 is a cross-sectional view schematically illustrating the sorting channel 120 in the sperm sorting apparatus 10 according to some embodiments of the present invention. It should be noted that, behavioral tendencies of the high motility sperms HM and low motility and/or dead sperms LM will be described with the first portion 120a of the sorting channel 120. However, such behavioral tendencies can be also observed in the second portion 120b and the third portion 120c of the sorting channel 120. In addition, an arrow AR indicates a flow direction of the medium solution flow.

Referring to FIG. 2A and FIG. 3, in addition to the behavior tendency of swimming against the medium solution flow, the high motility sperms further tend to gather at a top surface, a sidewall and a bottom surface of the sorting channel 120 in certain condition. Specifically, the high motility sperms HM in the sperm sample has such behavior tendency (i.e., gathering at the top surface, the sidewall and the bottom surface of the sorting channel 120) while being in a segment of the sorting channel 120 having a width greater than a critical dimension. On the other hand, the low motility and/or dead sperms LM may be left in a central passage of the sorting channel 120, and may be carried to the waste chamber 110 (as described with reference to FIG. 2A) by the medium solution flowing through such central passage (as indicated by the arrow AR). Consequently, more of the high motility sperms HM may gather in the sorting channel 120, then extracted from the sorting channel 120. In some embodiments, the width W₁₂₀ₐ (even its minimum value) of the first portion 120a of the sorting channel 120 is controlled to be greater than the critical dimension, such that the high motility sperms HM tend to gather in the first portion 120a of the sorting channel 120. For instance, the critical dimension may range from about 200 μm to about 400 μm (e.g., substantially equal to about 400 μm), and the width W₁₂₀ₐ may range from 20 mm to 30 mm. Further, the width W_{120c} of the third portion 120c of the sorting channel 120 may also be greater than the critical dimension, such that a portion of the high motility sperms HM may gather at a top surface, a sidewall and a bottom surface of the third portion 120c. Similarly, the width W_{120b} at a certain section of the second portion 120b of the sorting channel 120 may be greater than the critical dimension as well, such that a portion of the high motility sperms HM may gather in the second portion 120b.

On the other hand, if a width of a segment of the sorting channel 120 through which the sperm sample flows is less than the critical dimension, the high motility sperms HM may otherwise gather at corners of such segment (e.g., a corner defined by a top surface and a sidewall of such segment and a corner defined by the sidewall and a bottom surface of such segment), and may not spread across the top surface, the sidewall and the bottom surface of such segment. Therefore, fewer high motility sperms HM may gather in such segment. If a width of the sorting channel 120 is mostly or completely less than the critical dimension, then an amount of the extracted high motility sperms HM may be significantly limited.

In some embodiments, a height Duo of the sorting channel 120 may be about 100 μm. Moreover, portions of the sorting channel 120 (including the first portion 120a, the second portion 120b and the third portion 120c) may have substantially identical height (i.e., the height D₁₂₀). In other words, both of the top surface and the bottom surface of the sorting channel 120 may be substantially flat surfaces.

The sperm sorting apparatus 10 as described utilizes both of a behavioral tendency that the high motility sperms swim against the medium solution flow and a behavioral tendency that the high motility sperms gather across the inner surface of the sorting channel 120, thus a great amount of high motility sperms can be extracted by using the sperm sorting apparatus 10. As a result, in addition to be applicable for the ICSI artificial fertilization method, the sperm sorting apparatus 10 may be further applicable for the WF artificial fertilization method. For instance, by inserting a 100 μl sperm sample with sperm concentration about 12 M/ml into the sperm sorting apparatus 10, about 186,000 high motility sperms can be extracted. Further, by such extraction, a content of high motility sperms in the sperm sample is significantly raised from about 43.1% to about 91.3%. As another example, by inserting a 100 μl sperm sample with sperm concentration about 20.2 M/ml into the sperm sorting apparatus 10, about 156,000 high motility sperms can be extracted. In addition, by such extraction, a content of high motility sperms in the sperm sample is significantly raised from about 39.6% to about 92.3%.

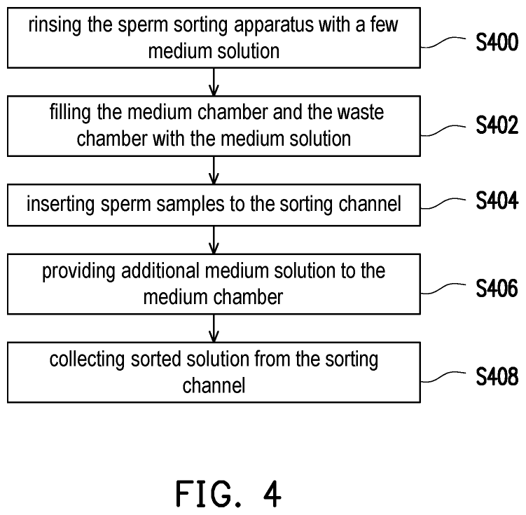

FIG. 4 is a flow diagram illustrating a sperm sorting method according to some embodiments of the present invention. Such sperm sorting method is performed by using the sperm sorting apparatus 10 as described above, and will be described with reference to FIG. 4 and FIG. 2A.

Referring to FIG. 4 and FIG. 2A, step S400 is performed, and the sperm sorting apparatus 10 is rinsed by a few medium solution. In some embodiments, the medium solution is inserted into the medium chamber 100, and flows to the waste chamber 110 through the micro-channels MC and the sorting channel 120, to rinse the whole sperm sorting apparatus 10. In addition, in some embodiments, the medium solution is Vitromed Sperm Wash.

At step S402, more medium solution is inserted into the medium chamber 100 and the waste chamber 110. In some embodiments, the medium chamber 100 and the waste chamber 110 are respectively inserted with 500 μl medium solution in the current step. Liquid levels in the medium chamber 100, the sorting channel 120 and the waste chamber 110 may become balanced after a period of time.

At step S404, a sperm sample is inserted into the sorting channel 120. The sperm sample may be inserted into the sorting channel 120 through the input/output hole H₁₂₀. In some embodiments, the sperm sample is a solution including sperm specimen and medium solution. In addition, in some embodiments, 100 μl of the sperm sample may be inserted into the sorting channel 120.

At step S406, additional medium solution is inserted into the medium chamber 100. Such additional medium solution may flow to the sorting channel 120 after the insertion, and form a medium solution flow in the sorting channel 120. In corresponding to the medium solution flow, the high motility sperms in the sorting channel 120 may exhibit the behavioral tendencies of swimming against the medium solution flow and gathering across the inner surface of the sorting channel 120. In some embodiments, the step of inserting additional medium solution into the medium chamber 100 is intermittently performed multiple times. For instance, 120 μl of medium solution is inserted into the medium chamber 100 for every 2 minutes in a total time of 10 minutes.

At step S408, a sorted solution is extracted from the sorting channel 120. A pipette may be used for extracting the sorted solution from the sorting channel 120 through the input/output hole H₁₂₀. The sorted solution includes the high motility sperms and the medium solution, and may include a few low motility and/or dead sperms. In some embodiments, the sorted solution of about 65 μl may be extracted from the sorting channel 120.

Up to here, the sperm sorting process according to some embodiments of the present invention has been completed. As described above, the high motility sperms at least mostly stay in the sorting channel 120 since being inserted into the sorting channel 120, while the low motility and/or dead sperms are carried to the waste chamber 110 along the medium solution flow. Subsequently, the sorted solution in the sorting channel 120 may be extracted from the sorting channel 120. In other words, a moving distance of the high motility sperms is short, thus the sorting can be performed in a short time, and energy consumption of the high motility sperms can be low. Moreover, since the sperm sample is directly inserted into the sorting channel 120, substantially all of the sperms in the sperm sample can be ensured to be sorted, thus a great sorting efficiency can be promised.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. A sperm sorting apparatus, comprising:
a medium chamber, configured to contain a medium solution;
a waste chamber, disposed aside the medium chamber, and configured to contain a residual solution obtained after sorting, wherein the residual solution includes low motility sperms and/or dead sperms; and
a sorting channel, extending between and communicated with the medium chamber and the waste chamber along a first direction, and configured to be inserted with a sperm sample, such that sperms in the sperm sample are sorted in corresponding to a medium solution flow entering the sorting channel from the medium chamber, wherein the sorting channel has a first portion and a second portion, the first portion is closer to the medium chamber than the second portion, a width of the first portion measured along a second direction is greater than a critical dimension, the second direction is intersected with the first direction, and the critical dimension ranges from 200 μm to 400 μm.

2. The sperm sorting apparatus according to claim 1, further comprising parallel micro-channels, extending along the second direction and disposed between the medium chamber and the sorting channel.

3. The sperm sorting apparatus according to claim 1, wherein the first portions of the sorting channel converges toward the medium chamber, a width of at least a section of the first portion measured along the second direction is greater than the critical dimension.

4. The sperm sorting apparatus according to claim 1, wherein the sorting channel is communicated to outside of the sperm sorting apparatus through an input/output hole.

5. The sperm sorting apparatus according to claim 4, wherein the input/output hole is communicated with the first portion of the sorting channel.

6. The sperm sorting apparatus according to claim 1, wherein the second portion of the sorting channel converges toward the waste chamber.

7. The sperm sorting apparatus according to claim 1, wherein an end portion of the sorting channel communicated with the waste chamber is a narrow channel, and a width of the sorting channel measured along the second direction is at its minimum at the narrow channel.

8. A sperm sorting method, comprising:
providing a sperm sorting apparatus, comprising a medium chamber, a waste chamber and a sorting channel extending between and communicated with the medium chamber and the waste chamber along a first direction, a width of at least a section of the sorting channel measured along a second direction is greater than a critical dimension, and the critical dimension ranges from 200 μm to 400 μm;
inserting a medium solution to the medium chamber and the waste chamber, till liquid levels in the medium chamber, the sorting channel and the waste chamber are balanced;
inserting a sperm sample into the sorting channel;
additionally inserting a medium solution into the medium chamber, such that the medium solution in the medium chamber flows into the sorting channel to form a medium solution flow, wherein sperms in the sperm sample are sorted in corresponding to the medium solution flow; and
extracting a sorted solution from the sorting channel.

9. The sperm sorting method according to claim 8, further comprising: rinsing the medium chamber, the sorting channel and the waste chamber with an additional medium solution before the step of inserting the medium solution to the medium chamber and the waste chamber.

10. The sperm sorting method according to claim 8, further comprising: intermittently repeating the step of additionally inserting a medium solution into the medium chamber before the step of extracting the sorted solution from the sorting channel.
